(19) United States
(12) Patent Application Publication
(10) Pub. No.: US 2024/0277670 A1
(43) Pub. Date: Aug. 22, 2024

(54) SAFE USE OF MMP-12 INHIBITOR

(71) Applicants: Yuhua LI, Newark, DE (US); Foresee Pharmaceuticals Co., Ltd., Taipei City (TW)

(72) Inventors: Yuhua LI, Newark, DE (US); Benjamin CHIEN, Newark, DE (US)

(21) Appl. No.: 18/568,043

(22) PCT Filed: Jun. 7, 2022
(86) PCT No.: PCT/US2022/072790

§ 371 Date: Dec. 7, 2023

Related U.S. Application Data
(60) Provisional application No. 63/208,273, filed on Jun. 8, 2021.

(51) Int. Cl. A61K 31/4178; A61K 9/16; A61K 9/48

(52) U.S. Cl. CPC A61K 31/4178; A61K 9/1652; A61K 9/4808

(57) ABSTRACT

Methods of safely administrating a matrix metalloproteinase 12 (MMP-12) inhibitor by oral administration are described. Also described are methods for providing clinically proven safe treatment of asthma, chronic obstructive pulmonary disease (COPD), or pulmonary fibrosis, by oral administration of an MMP-12 inhibitor.

### TECHINICAL FIELD

The application relates to methods of safely administrating a matrix metalloproteinase 12 (MMP-12) inhibitor and methods of providing clinically proven safe treatment of asthma, chronic obstructive pulmonary disease (COPD), and pulmonary fibrosis, by oral administration of a matrix metalloproteinase 12 (MMP-12) inhibitor.

### BACKGROUND

Matrix metalloproteinases (MMPs) comprise a family of over twenty zinc-dependent proteases and exhibit a wide range of biological activities. As a class, MMPs show structural similarities and share an array of common substrates, however, individual MMPs have distinct functions depending on their environment.

Among the MMP family, MMP-12 is a macrophage elastase, which is predominantly detected in alveolar macrophages. Besides, MMP-12 is produced by bronchial epithelial cells and airway smooth muscle cells. In both animals and humans, this protease is involved in type 2 inflammation as well as in tissue remodeling through its ability to turnover elastin during the whole life cycle. Accumulating evidence suggested the involvement of MMP-12 in the pathophysiology of chronic inflammatory airway diseases. In patients with asthma, chronic obstructive pulmonary disease (COPD), and/or pulmonary fibrosis, the extent of airway remodeling and/or disease severity correlate with gene-expression, local airway concentrations, and/or activities of MMP-12.

Asthma, COPD, and pulmonary fibrosis are the prevalent lung disorders worldwide. They are characterized by airflow obstruction and changes in airway architecture. It would be desirable, therefore, to provide new treatments with MMP-12 inhibitors which have their effect without serious adverse effects. In particular, for the treatment of asthma, COPD, and pulmonary fibrosis, it would be desirable to use MMP-12 inhibitors which can be administered orally and achieve therapeutically relevant exposure with minimal adverse effects.

### BRIEF SUMMARY

Disclosed herein are methods of safely administrating an MMP-12 inhibitor to subjects, including clinically proven safe treatment of asthma, COPD, or pulmonary fibrosis.

In one general aspect, the described herein is a method of safely administrating a compound of formula (I):

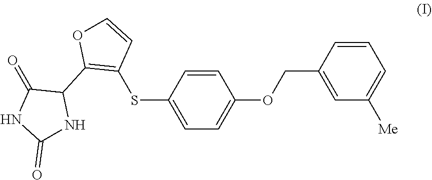 [SMILES: Cc1cccc(COc2ccc(Sc3ccoc3C3NC(=O)NC3=O)cc2)c1]

or a pharmaceutically acceptable salt thereof, to a human subject in need thereof, comprising orally administering to the subject a pharmaceutical composition comprising the compound of formula (I) or the pharmaceutically acceptable salt thereof and a cyclodextrin.

In some embodiments, a total dosage of the compound of formula (I) or the pharmaceutically acceptable salt thereof administered is about 25 mg to about 600 mg per administration.

In some embodiments, a total dosage of the compound of formula (I) or the pharmaceutically acceptable salt thereof administered per administration is about 25 mg, 50 mg, 100 mg, 150 mg, 200 mg, 250 mg, 300 mg, 350 mg, 400 mg, 450 mg, 500 mg, 550 mg, or 600 mg, or any dosage in between.

In some embodiments, the pharmaceutical composition is orally administered once per day, twice per day, or three times per day.

In some embodiments, the pharmaceutical composition is orally administered once per day. In such embodiments, the total dosage of the compound of formula (I) or the pharmaceutically acceptable salt thereof administered per day is about 25 mg, 50 mg, 100 mg, 150 mg, 200 mg, 250 mg, 300 mg, 350 mg, 400 mg, 450 mg, 500 mg, 550 mg, or 600 mg, or any dosage in between.

In some embodiments, the pharmaceutical composition is orally administered twice per day. In such embodiments, the total dosage of the compound of formula (I) or the pharmaceutically acceptable salt thereof administered per administration is about 50 mg, 100 mg, 150 mg, 200 mg, 250 mg, 300 mg, 350 mg, 400 mg, 450 mg, 500 mg, 550 mg, or 600 mg, or any dosage in between.

In some embodiments, when the pharmaceutical composition is orally administered twice per day, the total dosage of the compound of formula (I) or the pharmaceutically acceptable salt thereof administered per day is about 100 mg, 200 mg, 300 mg, 400 mg, 500 mg, 600 mg, 700 mg, 800 mg, 900 mg, 1000 mg, 1100 mg, or 1200 mg, or any dosage in between.

In some embodiments, the pharmaceutical composition is orally administered three times per day. In such embodiments, the total dosage of the compound of formula (I) or the pharmaceutically acceptable salt thereof administered per administration is about 50 mg, 100 mg, 150 mg, 200 mg, 250 mg, 300 mg, 350 mg, 400 mg, 450 mg, 500 mg, 550 mg, or 600 mg, or any dosage in between.

In some embodiments, when the pharmaceutical composition is orally administered three times per day, the total dosage of the compound of formula (I) or the pharmaceutically acceptable salt thereof administered per day is about 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600, 1700 mg, or 1800 mg, or any dosage in between.

In some embodiments, the administration of the pharmaceutical composition does not result in a serious adverse effect. In certain embodiments, the serious adverse effect comprises severe fatigue, allergic reactions, and arthralgia.

In some embodiments, the administration of the pharmaceutical composition does not result in clinically significant changes from the predose baseline in a laboratory assessment, a vital sign or an electrocardiogram (ECG).

In some embodiments, the human subject is in need of a treatment of asthma, chronic obstructive pulmonary disease (COPD), or pulmonary fibrosis.

In another general aspect, the invention relates to a method of treating a disease in a human subject in need thereof, the method comprising orally administering to the subject a pharmaceutical composition comprising a cyclodextrin and a compound of formula (I):

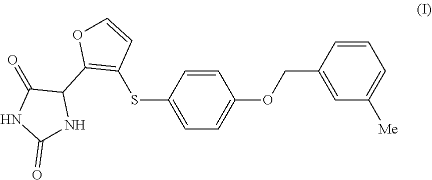 [SMILES: Cc1cccc(COc2ccc(Sc3ccoc3C3NC(=O)NC3=O)cc2)c1]

or a pharmaceutically acceptable salt thereof, wherein a total dosage of the compound of formula (I) or the pharmaceutically acceptable salt thereof administered is about 25 mg to about 600 mg per administration, and the disease is selected from the group consisting of asthma, chronic obstructive pulmonary disease (COPD), and pulmonary fibrosis.

In some embodiments, the treatment is a clinically proven safe treatment.

In some embodiments, the pharmaceutical composition is orally administered once per day, twice per day, or three times per day.

In some embodiments, the pharmaceutical composition is orally administered once per day. In such embodiments, the pharmaceutical composition is administered to the subject in an amount sufficient to provide about 25 mg per day, 50 mg per day, 100 mg per day, 200 mg per day, 300 mg per day, 400 mg per day, 500 mg per day, or 600 mg per day of the compound of formula (I) or a pharmaceutically acceptable salt.

In some embodiments, the pharmaceutical composition is orally administered twice per day. In such embodiments, the pharmaceutical composition is administered to the subject twice per day in an amount sufficient to provide about 100 mg per day, 200 mg per day, 300 mg per day, 400 mg per day, 500 mg per day, 600 mg per day, 700 mg per day, 800 mg per day, 900 mg per day, 1000 mg per day, 1100 mg per day, or 1200 mg per day of the compound of formula (I) or a pharmaceutically acceptable salt.

In some embodiments, the pharmaceutical composition is orally administered three times per day. In such embodiments, the pharmaceutical composition is administered to the subject three times per day in an amount sufficient to provide about 150 mg per day, 200 mg per day, 300 mg per day, 400 mg per day, 500 mg per day, 600 mg per day, 700 mg per day, 800 mg per day, 900 mg per day, 1000 mg per day, 1100 mg per day, 1200 mg per day, 1300 mg per day, 1400 mg per day, 1500 mg per day, 1600 mg per day, 1700 mg per day, or 1800 mg per day of the compound of formula (I) or a pharmaceutically acceptable salt.

The details of one or more embodiments of the invention are set forth in the description below. Other features and advantages will be apparent from the following detailed description, and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows a diagrammatic representation of the study design of a clinical study on single ascending dose using the compound of formula (I).

FIG. 2 shows maximum plasma concentration (Cₘₐₓ) of the compound of formula (I) versus dose in the single ascending study.

FIG. 3 shows area under curve of plasma concentration (AUCₜ) of the compound of formula (I) versus dose in the single ascending study.

FIG. 4 shows a diagrammatic representation of the study design of a clinical study on multiple ascending dose and food effect part using the compound of formula (I).

FIG. 5A shows mean plasma concentration of the compound of formula (I) versus time on Day 1 in the multiple ascending dose study, and FIG. 5B shows mean plasma concentration of the compound of formula (I) versus time on Day 8 in the multiple ascending dose study.

FIG. 6 shows mean plasma concentration of the compound of formula (I) versus time in the food effect study.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

### Definitions

It must be noted that as used herein and in the appended claims, the singular forms “a,” “an,” and “the” include plural reference unless the context clearly dictates otherwise.

As used herein, the term “about” preceding a numerical value or a series of numerical values means ±10% of the numerical value unless otherwise indicated. For example, “about 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, unless otherwise noted, the term “clinically proven” (used independently or to modify the term “safe”) shall mean that it has been proven by a clinical study in human subjects wherein the clinical study has met the approval standards of U.S. Food and Drug Administration, European Medicines Evaluation Agency (EMEA), or a corresponding national regulatory agency. In one embodiment of the application, the clinical study is a first-in-human, Phase I, randomized, double-blind, placebo-controlled, single ascending dose study of the compound of formula (I), which is an MMP-12 inhibitor, in healthy human subjects. In another embodiment of the application, the clinical study is a Phase I, randomized, double-blind, placebo-controlled, multiple ascending dose and food effect study of the compound of formula (I) in healthy human subjects.

As used herein, the phrases “adverse event (AE),” “treatment-emergent adverse event,” “adverse reaction,” and “adverse effect” mean any harm, unfavorable, unintended or undesired sign or outcome associated with or caused by administration of a pharmaceutical composition or therapeutic. However, abnormal values or observations are not reported as adverse events unless considered clinically significant by an investigator or a medical doctor.

As used herein, the phrases “serious adverse event (SAE)” and “serious adverse effect” mean any adverse event that is serious, as defined by the Food and Drug Administration (FDA) Code of Federal Regulations (CFR), Chapter 21. A SAE can be any AE or suspected adverse reaction that in the view of an investigator or a medical doctor, results in any of the following outcomes: death, a life threatening adverse event, inpatient hospitalization or prolongation of existing hospitalization, a persistent or significant incapacity or substantial disruption of the ability to conduct normal life functions, or a congenital anomaly/birth defect. Important medical events that may not result in death, be life threatening, or require hospitalization may be considered serious when, based upon appropriate medical judgment, they may jeopardize the patient or subject and may require medical or surgical intervention to prevent one of the outcomes listed in the above definition. Examples of such medical events include, but are not limited to, severe fatigue, allergic reactions, and arthralgia.

As used herein, when referring to safety assessment of the administration of the MMP-12 inhibitor, “clinically significant changes” means clinically apparent changes as determined by a medical doctor or an investigator using standard acceptable to those of ordinary skill in the art. When the harm or undesired outcome of adverse events reaches such a level of severity, a regulatory agency can deem the pharmaceutical composition or therapeutic unacceptable for the proposed use. Such changes can be measured by physical examination, such as examination of respiratory, cardiovascular, and gastrointestinal systems; laboratory assessments, such as hematology, blood chemistry, urinalysis, virus serology, urine drug screen, alcohol breath test, cotinine test, follicle stimulating hormone (FSH) and urine pregnancy test; vital signs, such as body temperature, respiratory rate, blood pressure and heart rate; and electrocardiogram (ECG) monitoring, including 12 lead safety ECGs.

As used herein, “treatment” or “treat” refers to the treatment of a disease, disorder, or medical condition (such as a gastrointestinal inflammatory disease), in a patient, such as a mammal (particularly a human) which includes one or more of the following:
(a) preventing the disease, disorder, or medical condition from occurring, i.e., preventing the reoccurrence of the disease or medical condition or prophylactic treatment of a patient that is pre-disposed to the disease or medical condition;
(b) ameliorating the disease, disorder, or medical condition, i.e., eliminating or causing regression of the disease, disorder, or medical condition in a patient, including counteracting the effects of other therapeutic agents;
(c) suppressing the disease, disorder, or medical condition, i.e., slowing or arresting the development of the disease, disorder, or medical condition in a patient; or
(d) alleviating the symptoms of the disease, disorder, or medical condition in a patient.

The terms “efficacy” and “effective” as used herein in the context of a dose, dosage regimen, treatment or method refer to the effectiveness of a particular dose, dosage or treatment regimen. Efficacy can be measured based on change in the course of the disease in response to an agent of the present invention. For example, the compound of formula (I) can be administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, the compound of formula (I) can be administered to achieve an improvement in a subject's condition related to asthma, chronic obstructive pulmonary disease (COPD), or pulmonary fibrosis.

The term “therapeutically effective amount” means an amount sufficient to effect treatment when administered to a patient in need of treatment.

### Methods of Safe Administration

In one general aspect, the invention relates to a method of safely administrating a compound of formula (I):

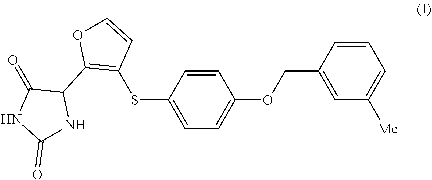 [SMILES: Cc1cccc(COc2ccc(Sc3ccoc3C3NC(=O)NC3=O)cc2)c1]

or a pharmaceutically acceptable salt thereof, to a human subject in need thereof, comprising orally administering to the subject a pharmaceutical composition comprising the compound of formula (I) or the pharmaceutically acceptable salt thereof and a cyclodextrin.

According to the embodiments of the invention, the compound of formula (I) has activity as an MMP-12 inhibitor. The compound of formula (I), its synthesis, biologic activities, uses or other related information thereof are described, for example, in U.S. Patent Application Publication No. US 2006/0041000, published on Feb. 23, 2006, the content of which is hereby incorporated by reference in its entirety. In addition, the pharmaceutical compositions comprising the compound of formula (I) are described, for example, in International Patent Application Publication No. WO 2018/035459, published on Feb. 22, 2018, the content of which is hereby incorporated by reference in its entirety.

The total dosage of the compound of formula (I) or the pharmaceutically acceptable salt thereof per administration is selected so as to provide safe/effective administration and/or safe/effective treatment as determined in clinical trials. According to embodiments of the invention, a total dosage of the compound of formula (I) or the pharmaceutically acceptable salt thereof administered is from about 25 mg to about 600 mg per administration, for example, 25 mg, 50 mg, 100 mg, 150 mg, 200 mg, 250 mg, 300 mg, 350 mg, 400 mg, 450 mg, 500 mg, 550 mg, or 600 mg, or any dosage in between.

In preferred embodiments, the total dosage of the compound of formula (I) or the pharmaceutically acceptable salt thereof administered per administration is from about 50 mg to about 450 mg, for example, 50 mg, 100 mg, 150 mg, 200 mg, 250 mg, 300 mg, 350 mg, 400 mg, or 450 mg, or any dosage in between.

In another preferred embodiments, the total dosage of the compound of formula (I) or the pharmaceutically acceptable salt thereof administered per administration is from about 100 mg to about 400 mg, for example, 100 mg, 150 mg, 200 mg, 250 mg, 300 mg, 350 mg, or 400 mg, or any dosage in between.

In some embodiments, the pharmaceutical composition is administered to the subject in an amount sufficient to provide from about 25 mg per day to about 1800 mg per day, for example, 25 mg, 50 mg, 75 mg, 100 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, or 1800 mg, or any dosage in between, of the compound of formula (I) or a pharmaceutically acceptable salt thereof.

In some embodiments, the pharmaceutical composition is administered to the subject in an amount sufficient to provide from about 100 mg per day to about 1200 mg per day, for example, 100 mg, 200 mg, 300 mg, 400 mg, 500 mg, 600 mg, 700 mg, 800 mg, 900 mg, 1000 mg, 1100 mg, or 1200 mg, or any dosage in between, of the compound of formula (I) or a pharmaceutically acceptable salt thereof. In some embodiments, the pharmaceutical composition is administered to the subject in an amount sufficient to provide from about 150 mg per day to about 1800 mg per day, for example, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600, 1700 mg, or 1800 mg, or any dosage in between, of the compound of formula (I) or a pharmaceutically acceptable salt thereof.

According to the embodiments of the invention, the pharmaceutical composition can be administered once per day, twice per day, three times per day, once per week, twice per week, etc.

In some embodiments, the pharmaceutical composition is orally administered once per day. In such embodiments, the total dosage of the compound of formula (I) or the pharmaceutically acceptable salt thereof administered per day is from about 25 mg to about 600 mg, for example, 25 mg, 50 mg, 100 mg, 150 mg, 200 mg, 250 mg, 300 mg, 350 mg, 400 mg, 450 mg, 500 mg, 550 mg, or 600 mg, or any dosage in between.

In some embodiments, the pharmaceutical composition is orally administered once per day. In such embodiments, the total dosage of the compound of formula (I) or the pharmaceutically acceptable salt thereof administered per day is from about 50 mg to about 450 mg, for example, 50 mg, 100 mg, 150 mg, 200 mg, 250 mg, 300 mg, 350 mg, 400 mg, 450 mg, or any dosage in between.

In some embodiments, the pharmaceutical composition is orally administered twice per day. In such embodiments, the total dosage of the compound of formula (I) or the pharmaceutically acceptable salt thereof administered per administration is from about 50 mg to about 600 mg, for example, 50 mg, 100 mg, 150 mg, 200 mg, 250 mg, 300 mg, 350 mg, 400 mg, 450 mg, 500 mg, 550 mg, or 600 mg, or any dosage in between.

In some embodiments, the pharmaceutical composition is orally administered twice per day. In such embodiments, the total dosage of the compound of formula (I) or the pharmaceutically acceptable salt thereof administered per administration is from about 100 mg to about 400 mg, for example, 100 mg, 150 mg, 200 mg, 250 mg, 300 mg, 350 mg, or 400 mg, or any dosage in between.

In some embodiments, the pharmaceutical composition is orally administered twice per day. In such embodiments, the total dosage of the compound of formula (I) or the pharmaceutically acceptable salt thereof administered per day is from about 100 mg to about 1200 mg, for example, 100 mg, 200 mg, 300 mg, 400 mg, 500 mg, 600 mg, 700 mg, 800 mg, 900 mg, 1000 mg, 1100 mg, or 1200 mg, or any dosage in between.

In some embodiments, the pharmaceutical composition is orally administered twice per day. In such embodiments, the total dosage of the compound of formula (I) or the pharmaceutically acceptable salt thereof administered per day is from about 200 mg to about 800 mg, for example, 200 mg, 300 mg, 400 mg, 500 mg, 600 mg, 700 mg, or 800 mg, or any dosage in between.

In some embodiments, the pharmaceutical composition is orally administered three times per day. In such embodiments, the total dosage of the compound of formula (I) or the pharmaceutically acceptable salt thereof administered per administration is from about 50 mg to about 600 mg, for example, 50 mg, 100 mg, 150 mg, 200 mg, 250 mg, 300 mg, 350 mg, 400 mg, 450 mg, 500 mg, 550 mg, or 600 mg, or any dosage in between.

In some embodiments, the pharmaceutical composition is orally administered three times per day. In such embodiments, the total dosage of the compound of formula (I) or the pharmaceutically acceptable salt thereof administered per administration is from about 100 mg to about 400 mg, for example, 100 mg, 150 mg, 200 mg, 250 mg, 300 mg, 350 mg, or 400 mg, or any dosage in between.

In some embodiments, the pharmaceutical composition is orally administered three times per day. In such embodiments, the total dosage of the compound of formula (I) or the pharmaceutically acceptable salt thereof administered per day is from about 150 mg to about 1800 mg, for example, 150 mg, 200 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600, 1700 mg, or 1800 mg, or any dosage in between.

In some embodiments, the pharmaceutical composition is orally administered three times per day. In such embodiments, the total dosage of the compound of formula (I) or the pharmaceutically acceptable salt thereof administered per day is from about 300 mg to about 1200 mg, for example, 300 mg, 400 mg, 500 mg, 600 mg, 700 mg, 800 mg, 900 mg, 1000 mg, 1100 mg, or 1200 mg, or any dosage in between.

In some embodiments, the pharmaceutically acceptable salt of the compound of formula (I) means a salt that is acceptable for administration to a patient or a mammal, such as a human (e.g., salts having acceptable mammalian safety for a given dosage regime). Representative pharmaceutically acceptable salts include, but are not limited to, salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

In some embodiments, the compound of formula (I) is amorphous.

In some embodiments, the administration of the pharmaceutical composition does not result in a serious adverse effect. In certain embodiments, the serious adverse effect is severe fatigue, allergic reactions, or arthralgia.

In some embodiments, the administration of the pharmaceutical composition does not result in clinically significant changes from the predose baseline in a laboratory assessment, a vital sign or an electrocardiogram (ECG).

In certain embodiments, the laboratory assessment is selected from the group consisting of hematology, blood chemistry, urinalysis, virus serology, urine drug screen, alcohol breath test, cotinine test, follicle stimulating hormone (FSH) and urine pregnancy test.

In certain embodiments, the vital sign is selected from the group consisting of heart rate, body temperature, sitting systolic blood pressure (SBP), sitting diastolic blood pressure (DBP), standing SBP, and standing DBP.

In certain embodiments, the ECG is safety 12-lead ECGs.

According to embodiments of the invention, a variety of factors can be analyzed to determine by clinical trials such as those described herein, whether a particular dosage of the compound of formula (I) provides for safe oral administration. For example, safety of a certain dosage of orally administered MMP-12 inhibitor can be assessed by pharmacokinetic studies (e.g., an area under the concentration time curve (AUC), and a maximum concentration observed (Cₘₐₓ). The safety of orally administered MMP-12 inhibitor can also be monitored by physical examination of the subject; allergic reactions; electrocardiograms; clinical laboratory tests; vital signs; and monitoring of other adverse events.

In some embodiments, clinically proven safe administration of the compound of formula (I) is determined by assessing pharmacokinetic (PK) parameters, such as an area under the concentration time curve (AUC), and a maximum concentration observed (Cₘₐₓ), of the inhibitor in the plasma of the subject. Plasma samples are analyzed to determine concentrations of the inhibitor by any method known in the art in view of the present disclosure. The pharmacokinetic parameters are then analyzed, for example, by non-compartment analysis (NCA), to calculate pharmacokinetic parameters, such as AUC, Cₘₐₓ, terminal half-life (T_{1/2}), total systemic clearance over bioavailability (CL/F), and volume of distribution at terminal phase over bioavailability (V_{Z}/F). In particular, AUC can be area under the concentration-time curve from time 0 to the 12 hour time point (AUC₀₋₁₂), area under the concentration-time curve from time 0 to the 24 hour time point (AUC₀₋₂₄), area under the concentration-time curve from time 0 extrapolated to infinity (AUC_{0-inf}), area under the concentration-time curve from time 0 to the last observed non-zero concentration (t) (AUC₀₋ₜ), area under the concentration-time curve during a dosing interval (tau) at steady state (AUC₀₋ₜₐᵤ), or area under the concentration-time curve from time 0 to the last observed non-zero concentration (t) at steady-state (AUCₛₛ₀₋ₜ)

In some embodiments, the single administration of the pharmaceutical composition in an amount sufficient to provide from about 25 mg to about 600 mg of the compound of formula (I) or a pharmaceutically acceptable salt thereof achieves, in the plasma of the subject, a mean area under the concentration time curve from time 0 extrapolated to infinity (AUC_{0-inf}) of about 1680 ng hr/mL to about 26000 ng hr/mL.

In some embodiments, the single administration of the pharmaceutical composition in an amount sufficient to provide from about 25 mg to about 600 mg of the compound of formula (I) or a pharmaceutically acceptable salt thereof achieves, in the plasma of the subject, a mean area under the concentration time curve from time 0 to the last observed non-zero concentration (t) (AUC₀₋ₜ), of about 1580 ng.hr/mL to about 25400 ng.hr/mL.

In some embodiments, the single administration of the pharmaceutical composition achieves in an amount sufficient to provide from about 25 mg to about 600 mg of the compound of formula (I) or a pharmaceutically acceptable salt thereof, in the plasma of the subject, a mean maximum concentration observed (Cₘₐₓ) of not more than about 2570 ng/mL.

In some embodiments, the single administration of the pharmaceutical composition achieves a time to reach maximum plasma concentration (Tₘₐₓ) of about 1 hour to about 6 hours, preferably about 1 hour to about 3 hours.

In some embodiments, the single administration of the pharmaceutical composition achieves a mean terminal elimination half-life (T_{1/2}) of about 6 hours to about 7 hours, preferably about 6.2 hours to about 6.9 hours.

In some embodiments, the single administration of the pharmaceutical composition achieves a mean apparent total clearance (CL/F) of about 17.9 L/h to about 31.8 L/h.

In some embodiments, the single administration of the pharmaceutical composition achieves a mean apparent volume of distribution (Vz/F) of about 169 L to about 253 L.

In some embodiments, when the pharmaceutical composition is orally administered twice per day, and the total dosage of the compound of formula (I) or a pharmaceutically acceptable salt thereof administered per administration is from about 50 mg to about 600 mg, the administration of the pharmaceutical composition achieves, in the plasma of the subject, a mean area under the concentration time curve from time 0 extrapolated to infinity (AUC_{0-inf}) of about 3550 ng.hr/mL to about 36300 ng.hr/mL.

In some embodiments, when the pharmaceutical composition is orally administered twice per day, and the total dosage of the compound of formula (I) or a pharmaceutically acceptable salt thereof administered per administration is from about 50 mg to about 600 mg, the administration of the pharmaceutical composition achieves, in the plasma of the subject, a mean maximum concentration observed (Cₘₐₓ) of not more than about 3710 ng/mL.

In some embodiments, when the pharmaceutical composition is orally administered twice per day, the administration of the pharmaceutical composition achieves a time to reach maximum plasma concentration (Tₘₐₓ) of about 0.5 hour to about 6 hours, preferably about 1 hour to about 3 hours.

In some embodiments, when the pharmaceutical composition is orally administered twice per day, the administration of the pharmaceutical composition achieves a steady-state condition of the compound of formula (I) within 6 days after the administration.

In some embodiments, when the pharmaceutical composition is orally administered twice per day, the administration of the pharmaceutical composition achieves a mean terminal elimination half-life (T_{1/2}) of about 6 hours to about 9 hours, preferably about 6.6 hours to about 8.4 hours.

In some embodiments, when the pharmaceutical composition is orally administered twice per day, the administration of the pharmaceutical composition achieves a mean apparent total clearance (CL/F) of about 16.2 L/h to about 23.1 L/h.

In some embodiments, when the pharmaceutical composition is orally administered twice per day, the administration of the pharmaceutical composition achieves a mean apparent volume of distribution (Vz/F) of about 158 L to about 291 L.

In some embodiments, the human subject is in need of a treatment of a disease selected from the group consisting of chronic obstructive pulmonary disease (COPD), and pulmonary fibrosis.

In another general aspect, the invention relates to a method of treating a disease in a human subject in need thereof, the method comprising orally administering to the subject a pharmaceutical composition comprising a cyclodextrin and a compound of formula (I):

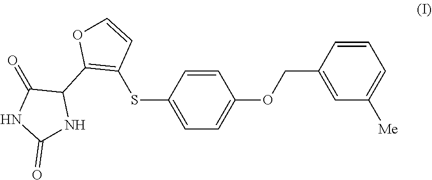 [SMILES: Cc1cccc(COc2ccc(Sc3ccoc3C3NC(=O)NC3=O)cc2)c1]

or a pharmaceutically acceptable salt thereof, wherein a total dosage of the compound of formula (I) or the pharmaceutically acceptable salt thereof administered is about 25 mg to about 600 mg per administration, and the disease is selected from the group consisting of asthma, chronic obstructive pulmonary disease (COPD), and pulmonary fibrosis.

In preferred embodiments, the total dosage of the compound of formula (I) or the pharmaceutically acceptable salt thereof administered per administration is from about 50 mg to about 450 mg, for example, 50 mg, 100 mg, 150 mg, 200 mg, 250 mg, 300 mg, 350 mg, 400 mg, or 450 mg, or any dosage in between.

In another preferred embodiments, the total dosage of the compound of formula (I) or the pharmaceutically acceptable salt thereof administered per administration is from about 100 mg to about 400 mg, for example, 100 mg, 150 mg, 200 mg, 250 mg, 300 mg, 350 mg, or 400 mg, or any dosage in between.

In some embodiments, the treatment is a clinically proven safe treatment.

In some embodiments, the pharmaceutical composition is orally administered once per day, twice per day, or three times per day.

In some embodiments, the pharmaceutical composition is orally administered once per day. In such embodiments, the pharmaceutical composition is administered to the subject in an amount sufficient to provide from about 25 mg per day to about 600 mg per day of the compound of formula (I) or a pharmaceutically acceptable salt, for example, about 25 mg per day, 50 mg per day, 100 mg per day, 200 mg per day, 300 mg per day, 400 mg per day, 500 mg per day, or 600 mg per day, or any dosage in between. In certain embodiments, the pharmaceutical composition is administered to the subject in an amount sufficient to provide from about 50 mg per day to about 450 mg per day of the compound of formula (I) or a pharmaceutically acceptable salt, for example, 50 mg per day, 100 mg per day, 150 mg per day, 200 mg per day, 250 mg per day, 300 mg per day, 350 mg per day, 400 mg per day, 450 mg per day, or any dosage in between.

In some embodiments, the pharmaceutical composition is orally administered twice per day. In such embodiments, the total dosage of the compound of formula (I) or the pharmaceutically acceptable salt thereof administered per administration is from about 50 mg to about 600 mg, for example, 50 mg, 100 mg, 150 mg, 200 mg, 250 mg, 300 mg, 350 mg, 400 mg, 450 mg, 500 mg, 550 mg, or 600 mg, or any dosage in between. In certain embodiments, the total dosage of the compound of formula (I) or the pharmaceutically acceptable salt thereof administered per administration is from about 100 mg to about 400 mg, for example, 100 mg, 150 mg, 200 mg, 250 mg, 300 mg, 350 mg, or 400 mg, or any dosage in between.

In some embodiments, the pharmaceutical composition is orally administered twice per day. In such embodiments, the pharmaceutical composition is administered to the subject twice per day in an amount sufficient to provide from about 100 mg per day to about 1200 mg per day of the compound of formula (I) or a pharmaceutically acceptable salt, for example, 100 mg per day, 200 mg per day, 300 mg per day, 400 mg per day, 500 mg per day, 600 mg per day, 700 mg per day, 800 mg per day, 900 mg per day, 1000 mg per day, 1100 mg per day, or 1200 mg per day, or any dosage in between. In certain embodiments, the pharmaceutical composition is administered to the subject twice per day in an amount sufficient to provide from about 200 mg per day to about 800 mg per day of the compound of formula (I) or a pharmaceutically acceptable salt, for example, 200 mg per day, 300 mg per day, 400 mg per day, 500 mg per day, 600 mg per day, 700 mg per day, or 800 mg per day, or any dosage in between.

In some embodiments, the pharmaceutical composition is orally administered three times per day. In such embodiments, the total dosage of the compound of formula (I) or the pharmaceutically acceptable salt thereof administered per administration is from about 50 mg to about 600 mg, for example, 50 mg, 100 mg, 150 mg, 200 mg, 250 mg, 300 mg, 350 mg, 400 mg, 450 mg, 500 mg, 550 mg, or 600 mg, or any dosage in between. In certain embodiments, the total dosage of the compound of formula (I) or the pharmaceutically acceptable salt thereof administered per administration is from about 100 mg to about 400 mg, for example, 100 mg, 150 mg, 200 mg, 250 mg, 300 mg, 350 mg, or 400 mg, or any dosage in between.

In some embodiments, the pharmaceutical composition is orally administered three times per day. In such embodiments, the pharmaceutical composition is administered to the subject three times per day in an amount sufficient to provide about 150 mg per day to about 1800 mg per day of the compound of formula (I) or a pharmaceutically acceptable salt, for example, 150 mg per day, 200 mg per day, 300 mg per day, 400 mg per day, 500 mg per day, 600 mg per day, 700 mg per day, 800 mg per day, 900 mg per day, 1000 mg per day, 1100 mg per day, 1200 mg per day, 1300 mg per day, 1400 mg per day, 1500 mg per day, 1600 mg per day, 1700 mg per day, or 1800 mg per day, or any dosage in between. In certain embodiments, the pharmaceutical composition is administered to the subject three times per day in an amount sufficient to provide about 300 mg per day to about 1200 mg per day of the compound of formula (I) or a pharmaceutically acceptable salt, for example, 300 mg per day, 400 mg per day, 500 mg per day, 600 mg per day, 700 mg per day, 800 mg per day, 900 mg per day, 1000 mg per day, 1100 mg per day, or 1200 mg per day, or any dosage in between.

In some embodiments, the compound of formula (I) is present in amorphous form.

### Pharmaceutical Compositions

According to the embodiments of the invention, the cyclodextrin for use in the pharmaceutical compositions herein is a water soluble unsubstituted or substituted alpha-cyclodextrin (ACD), beta-cyclodextrin (BCD), or gamma-cyclodextrin (GCD). In some embodiments, the beta-cyclodextrin is selected from the group consisting of methyl beta-cyclodextrin (MBCD), hydroxypropyl beta-cyclodextrin (HPBCD), and sulfobutylether beta-cyclodextrin (SBEBCD). In some embodiments, the beta-cyclodextrin is methyl beta-cyclodextrin or hydroxypropyl beta-cyclodextrin. In some embodiments, the gamma-cyclodextrin is hydroxypropyl gamma-cyclodextrin (HPGCD). In one preferred embodiment, the cyclodextrin is hydroxypropyl beta-cyclodextrin (HPBCD) or methyl beta-cyclodextrin (MBCD).

According to the embodiments of the invention, in the pharmaceutical composition, the weight ratio of the compound of formula (I) to the cyclodextrin is from 1:1 to 1:300, preferably from 1:1 to 1:50, and more preferably from 1:1 to 1:10.

In some embodiments, the weight ratio of the compound of formula (I) to the hydroxypropyl beta-cyclodextrin (HPBCD) is from 1:1 to 1:300, preferably from 1:1 to 1:50, and more preferably from 1:1 to 1:10.

In some embodiments, the compound of formula (I) and hydroxypropyl beta-cyclodextrin (HPBCD) are in the form of an amorphous solid dispersion (ASD).

According to the embodiments of the invention, the pharmaceutical composition of the invention typically contains a therapeutically effective amount of the compound of formula (I). Those skilled in the art will recognize, however, that a pharmaceutical composition can contain more than a therapeutically effective amount, e.g., bulk compositions, or less than a therapeutically effective amount, e. g., individual unit doses designed for multiple administration to achieve a therapeutically effective amount.

Typically, such pharmaceutical compositions contain from about 0.1% to about 95% by weight of the compound of formula (I); including from about 5% to about 70% by weight of the compound of formula (I).

The inventive pharmaceutical compositions comprising the compound of formula (I) and a cyclodextrin can further comprise a pharmaceutically acceptable carrier.

As used herein, the term “carrier” refers to any excipient, diluent, buffer, stabilizer, or other material well known in the art for pharmaceutical formulations. Pharmaceutically acceptable carriers in particular are non-toxic and should not interfere with the efficacy of the active ingredient. The pharmaceutically acceptable carriers include excipients and/or additives suitable for use in the pharmaceutical compositions known in the art, e.g., as listed in “Remington: The Science & Practice of Pharmacy”, 19th ed., Williams & Williams, (1995), and in the “Physician's Desk Reference”, 52nd ed., Medical Economics, Montvale, N.J. (1998), the disclosures of which are entirely incorporated herein by reference. Any conventional carrier or excipient may be used in the pharmaceutical compositions of the invention.

The choice of a particular carrier or excipient, or combinations of carriers or excipients, will depend on the mode of administration being used to treat a particular patient or type of medical condition or disease state. In this regard, the preparation of a suitable pharmaceutical composition for a particular mode of administration is well within the scope of those skilled in the pharmaceutical arts. Additionally, the carriers or excipients used in the pharmaceutical compositions of this invention are commercially available. By way of further illustration, conventional formulation techniques are described in Remington: The Science and Practice of Pharmacy, 20th Edition, Lippincott Williams & White, Baltimore, Maryland (2000); and H. C. Ansel et al., Pharmaceutical Dosage Forms and Drug Delivery Systems, 7th Edition, Lippincott Williams & White, Baltimore, Maryland (1999).

Representative examples of materials which can serve as pharmaceutically acceptable carriers include, but are not limited to, the following: sugars, such as lactose, glucose and sucrose; starches, such as corn starch and potato starch; cellulose, such as microcrystalline cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical compositions.

The pharmaceutical compositions of the disclosure are preferably packaged in a unit dosage form. The term “unit dosage form” refers to a physically discrete unit suitable for dosing a patient, i.e., each unit containing a predetermined quantity of active agent calculated to produce the desired therapeutic effect either alone or in combination with one or more additional units.

For example, such unit dosage forms may be capsules, tablets, pills, and the like, or unit packages suitable for parenteral administration.

According to the embodiments of the invention, suitable pharmaceutical compositions for oral administration can be in the form of capsules, tablets, pills, lozenges, cachets, dragees, powders, granules; or as a solution or a suspension in an aqueous or non-aqueous liquid; or as an oil-in-water or water-in-oil liquid emulsion; or as an elixir or syrup; and the like; each containing a predetermined amount of a compound of the present disclosure as an active ingredient.

When intended for oral administration in a solid dosage form (e.g., as capsules, tablets, pills and the like), the pharmaceutical compositions will typically comprise the active agent (the compound of formula (I)), the cyclodextrin, and one or more pharmaceutically-acceptable carriers. Optionally, such solid dosage forms may comprise: fillers or extenders, such as starches, microcrystalline cellulose, lactose, dicalcium phosphate, sucrose, glucose, mannitol, and/or silicic acid; binders, such as carboxymethylcellulose, alginates, gelatin, polyvinyl pyrrolidone, sucrose and/or acacia; humectants, such as glycerol; disintegrating agents, such as crosscarmellose sodium, agar-agar, calcium carbonate, potato or tapioca starch, alginic acid, certain silicates, and/or sodium carbonate; solution retarding agents, such as paraffin; absorption accelerators, such as quaternary ammonium compounds; wetting agents, such as cetyl alcohol and/or glycerol monostearate; absorbents, such as kaolin and/or bentonite clay; lubricants, such as talc, calcium stearate, magnesium stearate, solid polyethylene glycols, sodium lauryl sulfate, and/or mixtures thereof; coloring agents; and buffering agents.

Release agents, wetting agents, coating agents, sweetening, flavoring and perfuming agents, preservatives and antioxidants can also be present in the pharmaceutical compositions of the disclosure. Examples of pharmaceutically-acceptable antioxidants include: water-soluble antioxidants, such as ascorbic acid, cysteine hydrochloride, sodium bisulfate, sodium metabisulfate, sodium sulfite and the like; oil-soluble antioxidants, such as ascorbyl palmitate, butylated hydroxyanisole, butylated hydroxytoluene, lecithin, propyl gallate, alpha-tocopherol, and the like; and metal-chelating agents, such as citric acid, ethylenediamine tetraacetic acid, sorbitol, tartaric acid, phosphoric acid, and the like. Coating agents for tablets, capsules, pills and like, include those used for enteric coatings, such as cellulose acetate phthalate, polyvinyl acetate phthalate, hydroxypropyl methylcellulose phthalate, methacrylic acid, methacrylic acid ester copolymers, cellulose acetate trimellitate, carboxymethyl ethyl cellulose, hydroxypropyl methyl cellulose acetate succinate, and the like.

Pharmaceutical compositions of the invention can also be formulated to provide slow or controlled release of the active agent using, by way of example, hydroxypropyl methylcellulose in varying proportions; or other polymer matrices, liposomes and/or microspheres. In addition, the pharmaceutical compositions of the invention can optionally contain opacifying agents and may be formulated so that they release the active ingredient only, or preferentially, in a certain portion of the gastrointestinal tract, optionally, in a delayed manner. Examples of embedding compositions which can be used include polymeric substances and waxes. The active agent can also be in micro-encapsulated form, if appropriate, with one or more of the above-described excipients.

Suitable liquid dosage forms for oral administration include, by way of illustration, pharmaceutically-acceptable emulsions, microemulsions, solutions, suspensions, syrups and elixirs. Liquid dosage forms typically comprise the active agent and an inert diluent, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (esp., cottonseed, groundnut, corn, germ, olive, castor and sesame oils), oleic acid, glycerol, tetrahydrofuryl alcohol, polyethylene glycols and fatty acid esters of sorbitan, and mixtures thereof. Alternatively, certain liquid formulations can be converted, for example, by spray drying, to a powder, which is used to prepare solid dosage forms by conventional procedures.

Suspensions, in addition to the active ingredient, can contain suspending agents such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

According to embodiments of the invention, the pharmaceutical composition can be administered via a nasogastric tube. In such embodiments, the solid dosage form is opened up (for capsules and pills) or grounded into powders (for tablets), and then delivered as a suspension of solution in liquid, and the liquid dosage is delivered directly or diluted with another liquid.

### EXAMPLES

### Example 1: Study I: A Phase 1, Randomized, Double-Blinded, Placebo-Controlled, Single Ascending Dose Study of a Compound of Formula (I) in Healthy Subjects

This clinical study was the first in human study of a compound of formula (I) performed at Taipei Veterans General Hospital, Taipei, Taiwan. The purpose of this study was to assess the safety, tolerability and pharmacokinetics (PK) of the compound of formula (I) through single ascending dose administration in healthy subjects.

### Methods and Subjects

### Subject

Eligible participants were healthy male, aged between 18 and 65 years, and were in overall good physical and mental health conditions as determined by medical history and physical, vital signs, laboratory and electrocardiogramaxaminations. Other inclusion criteria consisted of a body mass index between 18 and 30 kg/m², a resting pulse rate between 50 and 100 beats per minute and resting blood pressure with a systolic blood pressure of ≤140 mmHg and a diastolic blood pressure of ≤90 mmHg. Male subjects had to use adequate contraception during and until three months after completion of the study. Main exclusion criteria consisted of: a history of alcohol or drug abuse, or being a current smoker, or using other nicotine products; the use of any prescription or non-prescription medications, herbal remedies, vitamins, or minerals within two weeks prior to the first dose (or within five half-lives prior to inclusion for any medications ingested, whichever was longer); or a positive test for hepatitis virus B or C, or human immunodeficiency virus, or a QT interval (by ECG examinations after the Bazett's correction) of more than 450 milliseconds.

### Single Ascending Dose (SAD)

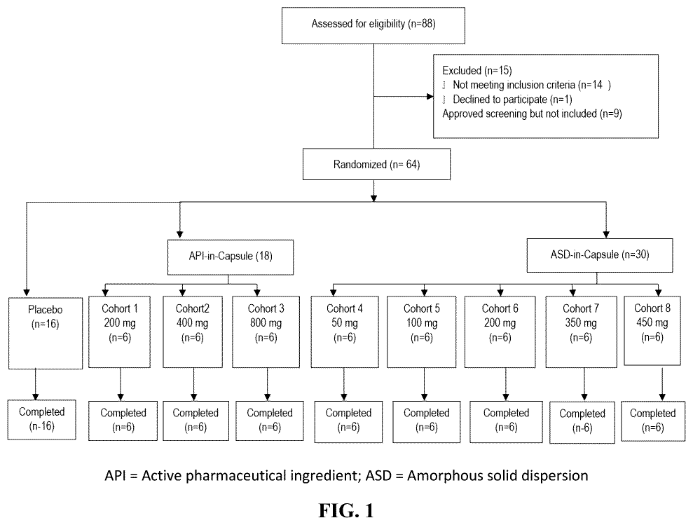

Eight cohorts of eight subjects each (six on active and two on placebo) were enrolled. Two different oral formulations of the compound of formula (I) were tested. The initial formulation was neat active pharmaceutical ingredient (API) filled in oral capsules of the compound of formula (I) (API-in-Capsule) which was tested at three dose levels: 200 mg, 400 mg, and 800 mg. Subsequently, an improved oral formulation was tested, the amorphous solid dispersion (ASD) of the compound of formula (I) (ASD-in-Capsule) at five dose levels: 50 mg, 100 mg, 200 mg, 350 mg, and 450 mg (FIG. 1).

API-in-Capsule cohorts: 200 mg, 400 mg, and 800 mg.

ASD-in-Capsule cohorts: 50 mg, 100 mg, 200 mg, 350 mg, and 450 mg.

### Study Agent

The ASD was prepared according to the procedure described in Example 6 of U.S. Pat. No. 10,532,102. The ASD consists of 25% the compound of formula (I) and 75% hydroxypropyl beta-cyclodextrin (HPBCD) by weight. In the study, matching placebo capsules were also tested.

### Study Assessments

### Safety and Tolerability Assessments

Safety and tolerability were assessed at pre-specified timepoints throughout the studies by AEs reporting, vital signs measurements, physical examination, laboratory tests, and ECG examinations.

### Pharmacokinetic Assessments

Blood samples (6 mL/sample) for plasma concentrations of the compound of formula (I) were drawn at 0.5, 1, 2, 3, 4, 6, 8, 12, 16, 20, 24, and 36 hours after each single dose. Within 15 minutes of collection, blood samples were centrifuged at 2000 rpm for 10 minutes at 4° C., and, subsequently, the plasma was aliquoted, frozen, and stored at −70° C. pending PK analyses.

The samples were subjected to liquid chromatography followed by analyses by tandem mass spectrometry. The lower limit of quantification for the compound of formula (I) was 5 ng/mL.

The plasma PK parameters were derived by non-compartmental analyses using Phoenix WinNonlin® version 6.3 or higher (Pharsight Corporation Inc., Mountain View, CA, USA). PK variables were summarized using arithmetic mean, standard deviation, median, minimum, maximum, the percent coefficient of variation, geometric mean, and a two-sided 95% confidence limit of the arithmetic mean and the geometric mean.

### Statistical Methods

Safety and tolerability data were evaluated for the treated population (all participants who took at least one dose of study medications). PK analyses were based on data from treated participants in whom at least one PK parameter could be calculated and who did not have any protocol violations that could interfere with these evaluations. Descriptive statistics were used to evaluate the safety and tolerability data and for plasma concentrations and PK parameters of the compound of formula (I).

Formal calculations of sample sizes were not performed. Based on the nature of the descriptive studies, the number of participants enrolled in each cohort was considered sufficient to meet the objectives of these phase I studies and to allow for assessments of PK parameters.

For the evaluations of food effect on the PK profile of the compound of formula (I), the analysis of variance (ANOVA) model included sequence, treatment, and period as fixed effects and subject nested within sequence as a random effect using the SAS® mixed model procedure. Each ANOVA included calculation of Least Squares Means (LSM), the difference between LSM under fed condition (Test) over fasted condition (Reference), and the standard error associated with this difference.

At each time-point, mean, median, standard deviation, minimum, maximum, number of available observations, and change from baseline were summarized by numeric parameters.

### Results and Discussions

### Subject Disposition and Blinding

In this study, 88 male subjects were screened and 64 were randomized (FIG. 1). All subjects completed the study. Overall, the subjects at the different dose levels were similar with regard to age, weight, height and BMI distribution (Table 1).

TABLE 1
Demographic and baseline characteristics of subjects participating in the SAD

| | | API-in- | API-in- | API-in- | ASD-in- | ASD-in- | ASD-in- | ASD-in- | ASD-in- |
|---|---|---|---|---|---|---|---|---|---|
| | | Capsule | Capsule | Capsule | Capsule | Capsule | Capsule | Capsule | Capsule |
| Variable | Placebo | 200 mg | 400 mg | 800 mg | 50 mg | 100 mg | 200 mg | 350 mg | 450 mg |
| Subjects, | 16 | 6 | 6 | 6 | 6 | 6 | 6 | 6 | 6 |
| n | | | | | | | | | |
| Age, | 28.8 | 26.3 | 28.5 | 29.5 | 28.5 | 28.0 | 28.3 | 31.0 | 25.8 |
| years | (6.28) | (2.66) | (6.69) | (8.34) | (2.51) | (4.47) | (5.57) | (5.44) | (2.79) |
| Male, | 16 | 6 | 6 | 6 | 6 | 6 | 6 | 6 | 6 |
| n | | | | | | | | | |
| Asian | 16 | 6 | 6 | 6 | 6 | 6 | 6 | 6 | 6 |
| race, n | | | | | | | | | |
| Height, | 173.19 | 170.95 | 174.33 | 173.83 | 175.77 | 176.27 | 177.58 | 173.5 | 169.0 |
| cm | (5.82) | (6.85) | (8.98) | (5.38) | (8.94) | (5.79) | (12.05) | (5.13) | (4.34) |
| Weight, | 70.7 | 63.83 | 64.67 | 69.17 | 74.53 | 71.5 | 73.2 | 69.2 | 62.83 |
| Kg | (9.72) | (7.89) | (7.0) | (7.86) | (16.22) | (8.09) | (14.46) | (6.56) | (5.23) |

Note:
Data are presented in mean (standard deviation) unless being specified.
API = Active pharmaceutical ingredient; ASD = Amorphous solid dispersion.

### Safety Results

All recorded AEs were self-limited and considered to be of mild intensity. No severe or serious AEs were reported. None of the subjects discontinued from the study due to an AE. No clinically relevant changes from baseline assessments were observed in weight, vital signs, physical examination, laboratory or ECG data after the administration of the compound of formula (I) at any time point.

Only one possibly related AE was reported during the SAD study (Table 2). This AE, mild diarrhea, was experienced by one subject following the API-in-Capsule 800 mg dosing and recovered spontaneously before the follow-up visit.

TABLE 2
Adverse events possibly-related to drug
administration reported during the SAD

| Adverse events | | | |
|---|---|---|---|
| description | Study | Dose level | Formulation |
| Diarrhea | Single ascending dose | 800 mg | API |

Note:
API = Active pharmaceutical ingredient; ASD = Amorphous solid dispersion.

### Pharmacokinetics Results

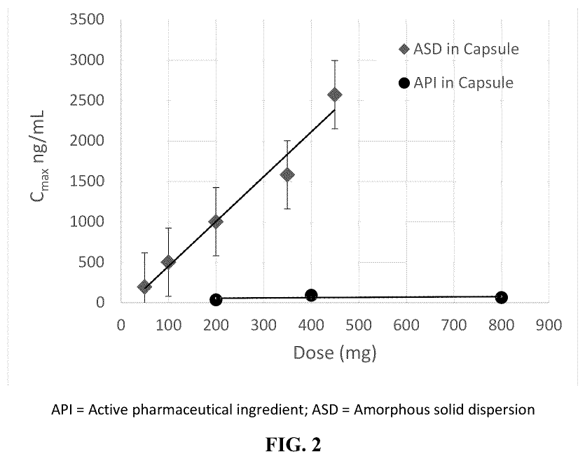

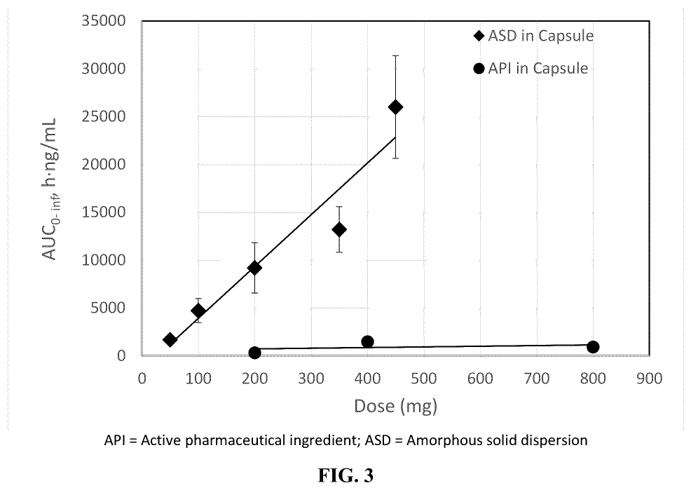

In the API-in-Capsule cohorts, median Tₘₐₓ was similar for the 200 and 400 mg doses, i.e., 5 and 6 hours, respectively, whereas median Tₘₐₓ appeared shorter (3.5 hours) for the 800 mg dose. Mean Cₘₐₓ, AUC₀₋ₜ, and AUC_{0-inf} increased in a more-than dose-proportional manner over the dose range of 200 to 400 mg, but decreased with the doses onward (Table 3, FIG. 2 and FIG. 3). In general, with the API-in-Capsule formulation, the exposure of the compound of formula (I) was low and inconsistent, with substantial inter-subject variability.

TABLE 3
Pharmacokinetic Parameters of Compound of Formula (I) after SAD

| | API-in-Capsule | | | | | |
|---|---|---|---|---|---|---|
| Parameter | 200 mg | | 400 mg | | 800 mg | |
| Cₘₐₓ (ng/mL) | 34.1 | (10.7) | 90.6 | (40.2) | 63.6 | (93.2) |
| Tₘₐₓ (h) | 5 | (2-6) | 6 | (4-12) | 3.5 | (3-12) |
| AUC₀₋ₜ (ng · h/mL) | 448 | (196) | 1380 | (771) | 1040 | (630) |

| | | | | |
|---|---|---|---|---|
| AUC_{0-inf} (ng · h/mL) | 313 | 1480 | (528) | 935 |
| λz (1/h) | 0.103 | 0.0748 | (0.01) | 0.0569 |
| T_{1/2} (h) | 6.73 | 9.42 | (1.5) | 12.5 |
| CL/F (L/h) | 638 | 293 | (87.9) | 856 |
| V_{z}/F (L) | 6200 | 3880 | (895) | 15500 |

ASD-in-Capsule

| | 50 mg | | 100 mg | | 200 mg | | 350 mg | | 450 mg | |
|---|---|---|---|---|---|---|---|---|---|---|
| Cₘₐₓ (ng/mL) | 193 | (25.2) | 498 | (113) | 1000 | (269) | 1580 | (309) | 2570 | (270) |
| Tₘₐₓ (h) | 2 | (1-3) | 1 | (1-3) | 2.5 | (1-3) | 1 | (1-3) | 2 | (1-6) |
| AUC₀₋ₜ(ng · h/mL) | 1580 | (402) | 4520 | (1160) | 8960 | (2540) | 12900 | (2260) | 25400 | (4980) |
| AUC_{0-inf} (ng · h/mL) | 1680 | (472) | 4720 | (1260) | 9180 | (2630) | 13200 | (2390) | 26000 | (5370) |
| λz (1/h) | 0.137 | (0.06) | 0.113 | (0.04) | 0.107 | (0.03) | 0.108 | (0.02) | 0.108 | (0.02) |
| T_{1/2} (h) | 6.21 | (3.23) | 6.68 | (2.03) | 6.88 | (1.9) | 6.69 | (1.6) | 6.68 | (1.37) |
| CL/F (L/h) | 31.8 | (8.93) | 22.5 | (5.98) | 23.4 | (6.84) | 23.2 | (3.79) | 17.9 | (3.56) |
| V_{z}/F (L) | 253 | (60.5) | 206 | (45.8) | 239 | (133) | 222 | (60.8) | 169 | (30.3) |

Note:
Data are in arithmetic mean (standard deviation) except for Tₘₐₓ which is reported in median (range).
The standard deviation was not calculated by some parameters because the total number was less than three.
API = Active pharmaceutical ingredient; ASD = Amorphous solid dispersion; AUC₀₋ₜ = AUC from time zero to time t of the last measurable concentration; AUC_{0-inf} = AUC from time zero to infinity; Cₘₐₓ = maximum observed plasma concentration; CL/F = apparent total body clearance; T_{1/2} = apparent terminal elimination half-life; Tₘₐₓ = time to reach maximum plasma concentration; V_{z}/F = apparent total volume of distribution; λz = terminal elimination rate constant.

In contrast, in the ASD-in-Capsule cohorts, median Tₘₐₓ ranged from 1 to 2.5 hours over the dose range of 50 to 450 mg after intake of single doses. Mean Cₘₐₓ, AUC₀₋ₜ and AUC_{0-inf} increased in a more-than-dose proportional manner over the dose range between of 50 to 100 mg. The drug exposure (mean Cₘₐₓ, AUC₀₋ₜ, and AUC_{0-inf}) increased in an approximately dose-proportional manner from 100 to 450 mg after intake of single doses of the compound of formula (I) (Table 3, FIG. 2 and FIG. 3). Dose-proportionality was found for Cₘₐₓ, AUC₀₋ₜ, and AUC_{0-inf} over the dose range of 100 to 450 mg after single doses of the compound of formula (I).

### Conclusions

The unique characteristic of this amorphous form of compound of formula (I) is that it not only significantly increased the bioavailability compared to the API-in-capsule, but also broke the exposure saturation set by the API itself. This is quite surprising and completely unexpected. Typically, due to the exposure saturation, one can only achieve the same bioavailability by simply dosing higher quantity of the API, and thus the program would have to be stopped at a point with AUC of about 1380 ng.h/mL with the API-in-capsule formulation (see FIG. 3). However, with the ASD-in-capsule formulation, a much higher AUC, an AUC of 25000 ng.h/mL, was achieved in a rather linear fashion (FIG. 2 and FIG. 3) when using the inventive ASD-in-capsule formulation.

Mean values of T_{1/2} were independent of the dose administered over the range of 50 to 450 mg after single oral doses of the compound of formula (I). Mean apparent total clearance (CL/F) and mean apparent volume of distribution (V_{Z}/F) of the compound of formula (I) were independent of the dose administered over the range of 100 to 350 mg.

The exposure of the compound of formula (I) appears substantially increased with an ASD-based formulation, which has been commonly used to improve oral absorption of drugs with low solubility (Jermain S V, et al., Int J Pharm, Jan. 15, 2018, 535(1-2):379-392). The highest exposure was observed in the 450 mg group (Table 3). Compared to the API-in-capsule formulation, the administration using the ASD-in-capsule formulation achieved higher and more consistent exposure, as well as lower inter-subject variability.

In summary, the exposure of the compound of formula (I) appears substantially increased with an ASD-based formulation. We also found that the ASD-in-capsule formulation resulted in shorter peak time, increased peak concentrations, and higher overall plasma exposure. Moreover, inter-subject variability of the PK parameters was lower in the ASD-in-capsule groups, and an overall dose-proportional increase was observed in the majority of the dose range tested. For example, at 200 mg dose, the ASD-in-capsule formulation resulted in 30 fold higher Cₘₐₓ and AUC_{0-inf} when compared to the API-in-capsule formulation. The percent coefficient of variation of these parameters also declined from approximately 30-40% to less than 30%. The decrease in inter-subject variability was more apparent at higher doses.

Across all dose levels that have been studied, the administration of the compound of formula (I) was safe and generally well-tolerated.

### Example 2: Study II: A Phase 1, Randomized, Double-Blinded, Placebo-Controlled, Multiple Dose Study of a Compound of Formula (I) in Healthy Subjects

The purpose of this study was to evaluate the safety, tolerability, and pharmacokinetics of the MMP-12 Inhibitor after multiple oral ascending dose administration, and to evaluate the effect of food after a single oral dose administration in healthy subjects. This phase I study consisted of 2 parts. The first part was a multiple ascending dose (MAD) part with a randomized, double-blind, placebo-controlled design in 3 treatment groups of 8 subjects (6 active; 2 placebo). The second part is a food effect (FE) part with a randomized, open-label, 2-period, 2-way crossover, single dose design in 8 subjects. This study was conducted at QPS-Netherlands, Groningen, the Netherlands.

### Methods and Subjects

### Subject

Eligible participants were healthy male and female of non-childbearing potential, aged between 18 and 65 years, and were in overall good physical and mental health conditions as determined by medical history and physical, vital signs, laboratory and electrocardiographic (ECG) examinations. Other inclusion criteria consisted of a body mass index between 18 and 30 kg/m², a resting pulse rate between 50 and 100 beats per minute and resting blood pressure with a systolic blood pressure of ≤140 mmHg and a diastolic blood pressure of ≤90 mmHg. Subjects had to use adequate contraception during and until three months after completion of the study. Main exclusion criteria consisted of: a history of alcohol or drug abuse, or being a current smoker, or using other nicotine products; the use of any prescription or non-prescription medications, herbal remedies, vitamins, or minerals within two weeks prior to the first dose (or within five half-lives prior to inclusion for any medications ingested, whichever was longer); or a positive test for hepatitis virus B or C, or human immunodeficiency virus, or a QT interval (by ECG examinations after the Bazett's correction) of more than 450 milliseconds.

### Part I—Multiple Ascending Dose (MAD)

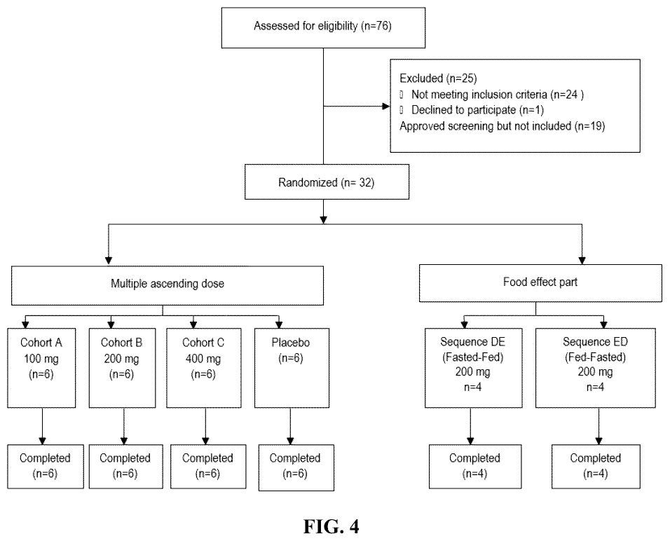

Three cohorts of eight subjects each (six on active and two on placebo) were enrolled and received the following treatments orally: 100 mg, 200 mg, and 400 mg ASD-in-Capsule b.i.d. (one dose on Days 1 and 8, twice-daily doses from Days 2 to 7) (FIG. 4).

### Part II—Food Effect (FE)

In this part, eight subjects received 200 mg ASD-in-Capsule orally as a single dose in a randomized way, once under fasting conditions and once after the intake of a high fat breakfast, with a wash-out period of one week between successive dosing (FIG. 4).

### Study Agent

The API-in-capsule and ASD-in-capsule were prepared the same way as in Example 1.

### Study Assessments

### Safety and Tolerability Assessments

Safety and tolerability were assessed at pre-specified timepoints throughout the studies by AEs reporting, vital signs measurements, physical examination, laboratory tests, and ECG examinations.

### Pharmacokinetic Assessments

Blood samples were collected post-dosing on Day 1 (at 0.5, 1, 2, 3, 4, 6, 8, 12, and 16 hours) and Day 8 (at 0.5, 1, 2, 3, 4, 6, 8, 10, 12, 16, 20, 24, 30, 36, and 48 hours) as well as immediately pre-morning dose on Days 2-7 and 2 hours post-morning dose on Days 4 and 6. Within 15 minutes of collection, blood samples were centrifuged at 2000 rpm for 10 minutes at 4° C., and, subsequently, the plasma was aliquoted, frozen, and stored at −70° C. pending PK analyses.

The samples were subjected to liquid chromatography followed by analyses by tandem mass spectrometry. The lower limit of quantification for the compound of formula (I) was 5 ng/mL.

The plasma PK parameters were derived by non-compartmental analyses using Phoenix® WinNonlin® version 6.3 or higher (Pharsight Corporation Inc., Mountain View, CA, USA). PK variables were summarized using arithmetic mean, standard deviation, median, minimum, maximum, the percent coefficient of variation, geometric mean, and a two-sided 95% confidence limit of the arithmetic mean and the geometric mean.

### Statistical Methods

Safety and tolerability data were evaluated for the treated population (all participants who took at least one dose of study medications). PK analyses were based on data from treated participants in whom at least one PK parameter could be calculated and who did not have any protocol violations that could interfere with these evaluations. Descriptive statistics were used to evaluate the for safety and tolerability data and for plasma concentrations and PK parameters of the compound of formula (I).

Formal calculations of sample sizes were not performed. Based on the nature of the descriptive studies, the number of participants enrolled in each cohort was considered sufficient to meet the objectives of these phase I studies and to allow for assessments of PK parameters.

For the evaluations of food effect on the PK profile of the compound of formula (I), the analysis of variance (ANOVA) model included sequence, treatment, and period as fixed effects and subject nested within sequence as a random effect using the SASR mixed model procedure. Each ANOVA included calculation of Least Squares Means (LSM), the difference between LSM under fed condition (Test) over fasted condition (Reference), and the standard error associated with this difference.

At each time-point, mean, median, standard deviation, minimum, maximum, number of available observations, and change from baseline were summarized by numeric parameters.

### Results and Discussions

### Subject Disposition and Blinding

In this study, 76 male and female subjects were screened, 24 of whom were randomized in the multiple dose part, while eight subjects were randomized in the food effect part of the study (FIG. 4). All subjects completed the study. Overall, the subjects at the different dose levels were similar with regard to age, weight, height and BMI distribution (Table 4).

TABLE 4
Demographic and baseline characteristics of
subjects participating in the MAD and FE

| | | Treatment | Treatment | Treatment | Treatment |
|---|---|---|---|---|---|
| Variable | Placebo | A | B | C | D/E |
| Subject n | 6 | 6 | 6 | 6 | 8 |
| Age, years | 30.5 | 32.3 | 34.0 | 33.2 | 44.5 |
| | (13.02) | (10.82) | (11.1) | (14.52) | (12.4) |
| Male, n | 6 | 6 | 6 | 5 | 7 |
| Black race, n | 0 | 2 | 3 | 0 | 0 |
| White race, n | 6 | 4 | 3 | 6 | 8 |
| Height, cm | 183.17 | 181.73 | 181.05 | 182.85 | 183.78 |
| | (9.96) | (4.36) | (7.95) | (7.78) | (7.09) |
| Weight, Kg | 82.87 | 81.95 | 69.4 | 73.93 | 85.33 |
| | (13.52) | (9.48) | (12.12) | (7.9) | (13.89) |

Note:
Data are presented in mean (standard deviation) unless being specified.
API = Active pharmaceutical ingredient; ASD = Amorphous solid dispersion.
Treatment A = oral dose of 100 mg ASD-in-Capsule;
Treatment B = oral dose of 200 mg ASD-in-Capsule;
Treatment C = oral dose of 400 mg ASD-in-Capsule;
Treatment D = single oral dose of 200 mg ASD-in-Capsule in fasted condition;
Treatment E = single oral dose of 200 mg ASD-in-Capsule in fed condition after intake of a high-fat, high calorie breakfast.

### Safety Results

All recorded AEs were self-limited and considered to be of mild intensity. No severe or serious AEs were reported. None of the subjects discontinued from the study due to an AE. No clinically relevant changes from baseline assessments were observed in weight, vital signs, physical examination, laboratory or ECG data after the administration of the compound of formula (I) at any time point.

In the MAD study, a total of seven AEs possibly related to treatment with the compound of formula (I) were reported (Table 5). Two of these events, comprising fatigue and cough, were reported by two subjects from the 100 mg group. The other five events were reported by three subjects from the 200 mg group: i.e., one subject with two episodes of eye irritation, and erythema, one subject with dizziness, and one subject with skin rash, respectively.

In the food effect part of the study, only two AEs possibly related to treatment with the compound of formula (I) were reported in one subject. Both events were headache, which occurred both under fed and fasted conditions.

TABLE 5
Adverse events possibly-related to drug administration
reported during the MAD and FE

| Adverse events | | Dose | |
|---|---|---|---|
| description | Study | Level | Formulation |
| Fatigue | Multiple ascending dose | 100 mg | ASD |
| Cough | Multiple ascending dose | 100 mg | ASD |
| Eye irritation | Multiple ascending dose | 200 mg | ASD |
| Erythema | Multiple ascending dose | 200 mg | ASD |
| Dizziness | Multiple ascending dose | 200 mg | ASD |
| Skin rash | Multiple ascending dose | 200 mg | ASD |
| Headache | Food effect fed status | 200 mg | ASD |
| Headache | Food effect fasting status | 200 mg | ASD |

Note:
ASD = Amorphous solid dispersion.

### Pharmacokinetics Results

The plasma concentration of the compound of formula (I) was determined in both the MAD and the FE parts of the study. In the MAD part, 18 subjects received the ASD in a dose-escalation scheme (100 mg, 200 mg, and 400 mg) and 6 subjects received placebo, so only the 18 subjects that received the ASD were included in the PK analysis. In the FE Part, 8 subjects received 200 mg ASD once under either fasted or fed condition (after the intake of a high-fat, high-caloric breakfast) and were included for the PK analysis.

Summary statistics of the PK parameters of the compound of formula (I) in the MAD and FE parts are summarized in Table 6 and Table 7, respectively. Statistical comparisons of the PK parameters in the FE part are presented in Table 8.

TABLE 6
Pharmacokinetic Parameters of Compound of Formula (I) after MAD

| | Treatment A (100 mg) | | Treatment B (200 mg) | | Treatment C (400 mg) | |
|---|---|---|---|---|---|---|
| Parameters | N | Mean (SD) | N | Mean (SD) | N | Mean (SD) |

Day 1

| | | | | | | | | | |
|---|---|---|---|---|---|---|---|---|---|
| Cₘₐₓ, ng/mL | 6 | 448 | (65.9) | 6 | 1200 | (338) | 6 | 2730 | (671) |
| Tₘₐₓ, h ^{a} | 6 | 1.00 | (0.50-2.00) | 6 | 1.02 | (1.00-4.00) | 6 | 1.00 | (1.00-3.00) |
| AUC₀₋₁₂, h · ng/mL | 6 | 2670 | (532) | 6 | 7810 | (1870) | 6 | 14600 | (4480) |
| AUC₀₋₂₄, h · ng/mL | 6 | 3290 | (593) | 6 | 9720 | (2320) | 6 | 18200 | (6520) |
| AUC₀₋ₜ, h · ng/mL | 6 | 3290 | (593) | 6 | 9710 | (2320) | 6 | 18200 | (6510) |
| AUC_{0-inf}, h · ng/mL | 6 | 3550 | (547) | 6 | 10400 | (2520) | 6 | 19900 | (7950) |
| Kel, 1/h | 6 | 0.106 | (0.0266) | 6 | 0.119 | (0.0243) | 6 | 0.113 | (0.0262) |
| t_{1/2}, h | 6 | 6.90 | (1.82) | 6 | 6.02 | (1.03) | 6 | 6.48 | (1.87) |
| CL/F, L/h | 6 | 28.7 | (3.96) | 6 | 20.3 | (5.48) | 6 | 24.0 | (12.1) |
| V_{z}/F, L | 6 | 291 | (107) | 6 | 176 | (58.0) | 6 | 212 | (86.6) |

Day 8

| | | | | | | | | | |
|---|---|---|---|---|---|---|---|---|---|
| AI | 6 | 1.59 | (0.284) | 6 | 1.57 | (0.236) | 6 | 1.40 | (0.249) |
| Cₘₐₓ, ng/mL | 6 | 624 | (141) | 6 | 1920 | (366) | 6 | 3710 | (932) |
| Tₘₐₓ, h ^{a} | 6 | 2.00 | (1.00-3.00) | 6 | 1.50 | (1.00-3.00) | 6 | 1.50 | (0.50-2.00) |
| Cₘᵢₙ, ng/mL | 6 | 156 | (27.4) | 6 | 524 | (196) | 6 | 1010 | (651) |
| AUC₀₋₁₂, h · ng/mL | 6 | 4380 | (543) | 6 | 13100 | (3440) | 6 | 24700 | (8350) |
| AUC_{0-inf}, h · ng/mL | 6 | 6180 | (889) | 6 | 19200 | (5970) | 6 | 36300 | (17900) |
| C_{avg}, ng/mL | 6 | 365 | (45.3) | 6 | 1090 | (287) | 6 | 2060 | (696) |
| Fl, % | 6 | 127 | (30.0) | 6 | 132 | (21.8) | 6 | 142 | (53.4) |
| Kel, 1/h | 6 | 0.0897 | (0.0276) | 6 | 0.0895 | (0.0204) | 6 | 0.115 | (0.0335) |
| t_{1/2}, h | 6 | 8.37 | (2.57) | 6 | 8.14 | (2.12) | 6 | 6.57 | (2.31) |
| CLss/F, L/h | 6 | 23.1 | (3.16) | 6 | 16.2 | (4.08) | 6 | 18.2 | (7.53) |
| V_{z}/F, L | 6 | 277 | (85.1) | 6 | 189 | (60.7) | 6 | 158 | (29.8) |
| RAUC1 | 6 | 1.68 | (0.327) | 6 | 1.68 | (0.207) | 6 | 1.69 | (0.114) |
| RAUC2 | 6 | 1.25 | (0.187) | 6 | 1.26 | (0.184) | 6 | 1.28 | (0.113) |

^{a} Median (range);
RAUC1: AUC₀₋₁₂ (Day 8)/AUC₀₋₁₂ (Day 1);
RAUC2: AUC₀₋₁₂ (Day 8)/AUC_{0-inf} (Day 1);
Mean: arithmetic mean;
SD: standard deviation;
Treatment A: 14 oral doses of 100 mg of the compound of formula (I) (n = 6) or placebo (n = 2) in 8 days;
Treatment B: 14 oral doses of 200 mg of the compound of formula (I) (n = 6) or placebo (n = 2) in 8 days;
Treatment C: 14 oral doses of 400 mg of the compound of formula (I) (n = 6) or placebo (n = 2) in 8 days;
On Day 1 and Day 8, the study drugs were given q.d. and on Day 2 through Day 7, the study drugs were given b.i.d. Reference source: Post-text Table 14.4.2

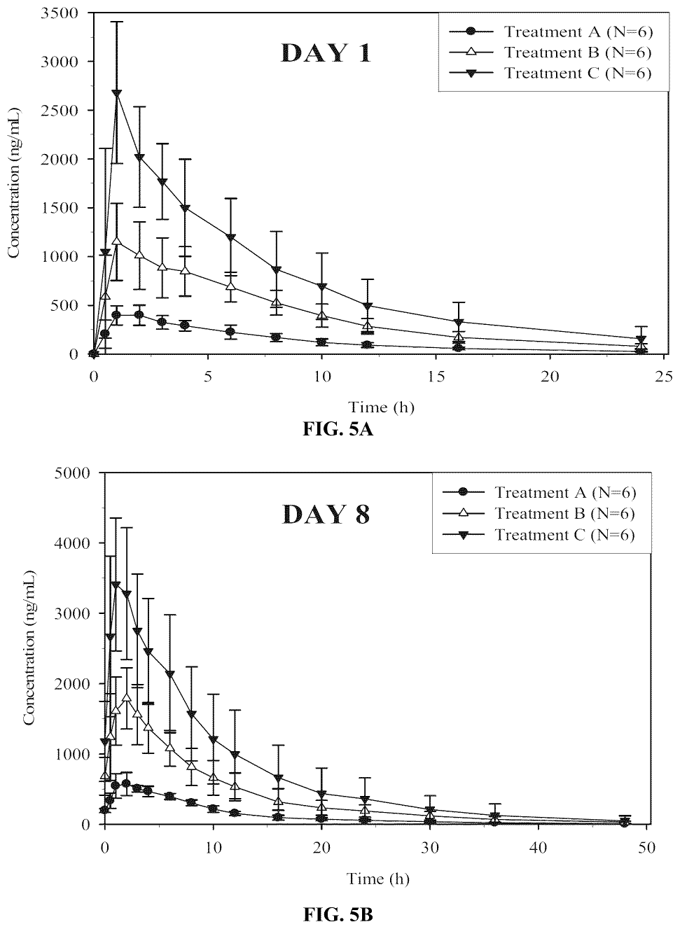

The peak exposure (Cₘₐₓ) and the systemic exposure (AUC) of the compound of formula (I) increased in a more-than-dose proportional manner from 100 to 200 mg and approximately in a dose-proportional manner from 200 to 400 mg after multiple doses for a week. Median Tₘₐₓ of the compound of formula (I) was similar among the three escalating dose levels both on Day 1 and Day 8 (see also, FIGS. 5A and 5B). T_{1/2} of the compound of formula (I) at single doses of 100, 200, and 400 mg, was 6.90, 6.02, and 6.48 hours on Day 1, respectively; the corresponding values after intake of multiple doses was 8.37, 8.14, and 6.57 hours on Day 8, respectively. CL/F was 28.7, 20.3, and 24.0 L/h after single doses at 100, 200, and 400 mg, respectively. The corresponding values after multiple doses were 23.1, 16.2, and 18.2 L/h, respectively. Similarly, estimated V/F was similar between Day 1 and Day 8. Steady state was achieved by Day 6 for all three dose levels. The AUC ratio of the compound of formula (I) (Day 8/Day 1) was approximately 1.7-fold after twice-daily administration at all three dose levels.

TABLE 7
Pharmacokinetic Parameters of Compound of Formula (I) after FE

| | Treatment D | | | Treatment E | | |
|---|---|---|---|---|---|---|
| | | Mean | | | Mean | |
| Parameters | N | (SD) | | N | (SD) | |
| Cₘₐₓ, ng/mL | 8 | 1050 | (270) | 8 | 787 | (204) |
| Tₘₐₓ, h ^{a} | 8 | 1.00 | (0.50-1.00) | 8 | 2.50 | (2.00-4.00) |
| AUC₀₋ₜ, | 8 | 6490 | (1880) | 8 | 6800 | (2060) |
| h · ng/mL | | | | | | |
| AUC_{0-inf}, | 8 | 6620 | (1850) | 8 | 6900 | (2070) |
| h · ng/mL | | | | | | |
| Kel, 1/h | 8 | 0.0796 | (0.0213) | 8 | 0.0917 | (0.0243) |
| t_{1/2}, h | 8 | 9.36 | (2.92) | 8 | 8.09 | (2.44) |
| CL/F, L/h | 8 | 32.3 | (8.90) | 8 | 32.3 | (13.5) |
| V_{z}/F, L | 8 | 446 | (189) | 8 | 383 | (211) |

^{a} Median (range);
Mean: arithmetic mean;
SD: standard deviation;
Treatment D: single oral dose of 200 mg of the compound of formula (I) under fasted condition;
Treatment E: single oral dose of 200 mg of the compound of formula (I) after intake of the high-fat, high-calorie breakfast (fed condition). Reference source: Post-text Table 14.4.3

TABLE 8
Statistical Comparisons of Pharmacokinetic Parameters of after FE

| | Treatment D | | Treatment E | | Treatment E/D |
|---|---|---|---|---|---|
| PK Parameter | N | GM | N | GM | GMR(90% CI) |
| Cₘₐₓ^{†} (ng/mL) | 8 | 1020 | 8 | 764 | 74.53 |
| | | | | | (66.33, 83.76) |
| AUC₀₋^{†} (h · ng/mL) | 8 | 6260 | 8 | 6480 | 103.42 |
| | | | | | (94.54, 113.14) |
| AUC_{0-inf}^{†} (h · ng/mL) | 8 | 6400 | 8 | 6580 | 102.76 |
| | | | | | (93.65, 112.76) |

^{†}Back-transformed least squares mean and confidence interval from ANOVA model performed on log-transformed values;
GM = Geometric least-squares mean;
GMR = Geometric least-squares mean ratio;
CI = Confidence interval;
GMR and 90% CI: Reported as percentage;
Treatment D: single oral dose of 200 mg of the compound of formula (I)under fasted condition;
Treatment E: single oral dose of 200 mg of the compound of formula (I)after intake of the high-fat, high-calorie breakfast (fed condition).

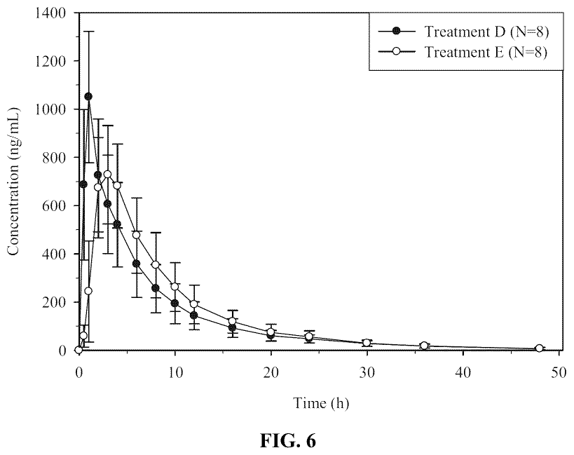

As indicated in Table 7 and Table 8, co-administering the compound of formula (I) with food appears to have no effect on overall exposure (similar AUCs were obtained with or without food), although the peak time increased from 1 to 2.5 hours and a lower peak concentration was observed (1050 ng/mL vs 787 ng/mL, see FIG. 6). T_{1/2} was 9.36 and 8.09 hours under fasted and fed conditions, respectively. CL/F was 32.3 L/h for both fasted and fed conditions. Vz/F was 446 L and 383 L under fasted and fed conditions, respectively. Because the compound of formula (I) is developed for chronic disease treatment, the minor shift in PK profile may not be significant to mandate dosing with or without food.

### Conclusions

Across all dose levels and dosing regimens that have been studied, the compound of formula (I) was safe and generally well-tolerated. There were only few AEs that were mild, short-lasting, and self-limited. There was no increase in frequency or intensity of AEs at higher dose levels. Our data extend and complement the scarce clinical studies with previously tested, often non-selective MMP inhibitors.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present inventions as defined by the specific description.

1. A method of safely administering a compound of formula (I) 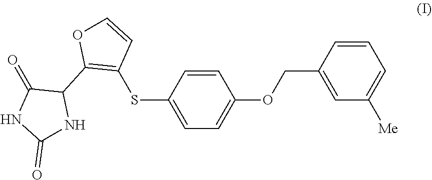 [SMILES: Cc1cccc(COc2ccc(Sc3ccoc3C3NC(=O)NC3=O)cc2)c1]
or a pharmaceutically acceptable salt thereof, to a human subject in need thereof, comprising orally administering to the subject a pharmaceutical composition comprising the compound of formula (I) or the pharmaceutically acceptable salt thereof and a cyclodextrin, wherein a total dosage of the compound of formula (I) or the pharmaceutically acceptable salt thereof administered is about 25 mg to about 600 mg per administration.

2. The method of claim 1, wherein the total dosage of the compound of formula (I) or the pharmaceutically acceptable salt thereof administered per administration is about 25 mg, 50 mg, 100 mg, 150 mg, 200 mg, 250 mg, 300 mg, 350 mg, 400 mg, 450 mg, 500 mg, 550 mg, or 600 mg, or any dosage in between.

3. The method of claim 1 or 2, wherein the pharmaceutical composition is orally administered once per day or twice per day.

4. The method of any one of claims 1 to 3, wherein the pharmaceutical composition is orally administered twice per day, and the total dosage of the compound of formula (I) or the pharmaceutically acceptable salt thereof administered per administration is about 50 mg, 100 mg, 150 mg, 200 mg, 250 mg, 300 mg, 350 mg, 400 mg, 450 mg, 500 mg, 550 mg, or 600 mg, or any dosage in between.

5. The method of any one of claims 1-4, wherein the pharmaceutical composition is orally administered twice per day, and the total dosage of the compound of formula (I) or the pharmaceutically acceptable salt thereof administered per day is about 100 mg, 200 mg, 300 mg, 400 mg, 500 mg, 600 mg, 700 mg, 800 mg, 900 mg, 1000 mg, 1100 mg, or 1200 mg, or any dosage in between.

6. The method of any one of claims 1 to 5, wherein the administration of the pharmaceutical composition does not result in a serious adverse effect.

7. The method of claim 6, wherein the serious adverse effect is selected from the group consisting of severe fatigue, allergic reactions, and arthralgia.

8. The method of any one of claims 1 to 7, wherein the administration of the pharmaceutical composition does not result in clinically significant changes from the predose baseline in a laboratory assessment, a vital sign or an electrocardiogram (ECG).

9. The method of any one of claims 1 to 3, wherein the pharmaceutical composition is orally administered once per day, and the administration of the pharmaceutical composition achieves. in the plasma of the subject, a mean area under the concentration time curve from time 0 extrapolated to infinity (AUC_{0-inf}) of about 1680 ng.hr/mL to about 26000 ng.hr/mL.

10. The method of any one of claims 1 to 3, wherein the pharmaceutical composition is orally administered once per day, and the administration of the pharmaceutical composition achieves, in the plasma of the subject, a mean maximum concentration observed (Cₘₐₓ) of not more than about 2570 ng/mL.

11. The method of any one of claims 1 to 3, wherein the pharmaceutical composition is orally administered once per day, and the administration of the pharmaceutical composition achieves a mean terminal elimination half-life (T_{1/2}) of about 6 hours to about 7 hours, preferably about 6.2 hours to about 6.9 hours.

12. The method of any one of claims 1 to 3, wherein the pharmaceutical composition is orally administered once per day, and the administration of the pharmaceutical composition achieves a time to reach maximum plasma concentration (Tₘₐₓ) of about 1 hour to about 6 hours, preferably about 1 hour to about 3 hours.

13. The method of any one of claims 1 to 3, wherein the pharmaceutical composition is orally administered once per day, and the administration of the pharmaceutical composition achieves a mean apparent total clearance (CL/F) of about 17.9 L/h to about 31.8 L/h.

14. The method of any one of claims 1 to 3, wherein the pharmaceutical composition is orally administered once per day, and the administration of the pharmaceutical composition achieves a mean apparent volume of distribution (V_{Z}/F) of about 169 L to about 253 L.

15. The method of any one of claims 1 to 5, wherein the pharmaceutical composition is orally administered twice per day, and the administration of the pharmaceutical composition achieves, in the plasma of the subject, a mean area under the concentration time curve from time 0 extrapolated to infinity (AUC_{0-inf}) of about 3550 ng.hr/mL. to about 36300 ng.hr/mL.

16. The method of any one of claims 1 to 5, wherein the pharmaceutical composition is orally administered twice per day, and the administration of the pharmaceutical composition achieves, in the plasma of the subject, a mean maximum concentration observed (Cₘₐₓ) of not more than about 3710 ng/mL.

17. The method of any one of claims 1 to 5, wherein the pharmaceutical composition is orally administered twice per day, and the administration of the pharmaceutical composition achieves, in the plasma of the subject, a steady-state condition of the compound of formula (I) within 6 days.

18. The method of any one of claims 1 to 5, wherein the pharmaceutical composition is orally administered twice per day, and the administration of the pharmaceutical composition achieves a mean terminal elimination half-life (T_{1/2}) of about 6 hours to about 9 hours, preferably about 6.6 hours to about 8.4 hours.

19. The method of any one of claims 1 to 5, wherein the pharmaceutical composition is orally administered twice per day, and the administration of the pharmaceutical composition achieves a time to reach maximum plasma concentration (Tₘₐₓ) of about 0.5 hour to about 6 hours, preferably about 1 hour to about 3 hours.

20. The method of any one of claims 1 to 5, wherein the pharmaceutical composition is orally administered twice per day, and the administration of the pharmaceutical composition achieves a mean apparent total clearance (CL/F) of about 16.2 L/h to about 23.1 L/h.

21. The method of any one of claims 1 to 5, wherein the pharmaceutical composition is orally administered twice per day, and the administration of the pharmaceutical composition achieves a mean apparent volume of distribution (V_{Z}/F) of about 158 L to about 291 L.

22. The method of claim 1, wherein the human subject is in need of a treatment of a disease selected from the group consisting of asthma, chronic obstructive pulmonary disease (COPD), and pulmonary fibrosis.

23. A method of treating a disease in a human subject in need thereof, the method comprising orally administering to the subject a pharmaceutical composition comprising a cyclodextrin and a compound of formula (I): 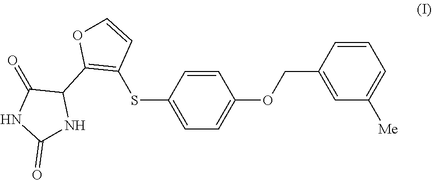 [SMILES: Cc1cccc(COc2ccc(Sc3ccoc3C3NC(=O)NC3=O)cc2)c1]
or a pharmaceutically acceptable salt thereof, wherein a total dosage of the compound of formula (I) or the pharmaceutically acceptable salt thereof administered is about 25 mg to about 600 mg per administration, and the disease is selected from the group consisting of asthma, chronic obstructive pulmonary disease (COPD), and pulmonary fibrosis.

24. The method of claim 23, wherein the total dosage of the compound of formula (I) or the pharmaceutically acceptable salt thereof administered per administration is about 25 mg, 50 mg, 100 mg, 150 mg, 200 mg, 250 mg, 300 mg, 350 mg, 400 mg, 450 mg, 500 mg, 550 mg, or 600 mg, or any dosage in between.

25. The method of claim 23 or 24, wherein the pharmaceutical composition is orally administered once per day or twice per day.

26. The method of any one of claims 23 to 25, wherein the pharmaceutical composition is orally administered twice per day, and the total dosage of the compound of formula (I) or the pharmaceutically acceptable salt thereof administered per administration is about 50 mg, 100 mg, 150 mg, 200 mg, 250 mg, 300 mg, 350 mg, 400 mg, 450 mg, 500 mg, 550 mg, or 600 mg, or any dosage in between.

27. The method of any one of claims 23 to 25, wherein the pharmaceutical composition is orally administered twice per day, and the total dosage of the compound of formula (I) or the pharmaceutically acceptable salt thereof administered per day is about 100 mg, 200 mg, 300 mg, 400 mg, 500 mg, 600 mg, 700 mg, 800 mg, 900 mg, 1000 mg, 1100 mg, or 1200 mg, or any dosage in between.

28. The method of any one of claims 1 to 27, wherein the composition comprises the compound of formula (I).

29. The method of claim 28, wherein the compound of formula (I) is amorphous.

30. The method of any one of claims 1 to 29, wherein the cyclodextrin is a hydroxypropyl beta-cyclodextrin (HPBCD).

31. The method of any one of claims 1 to 30, wherein a weight ratio of the compound of formula (I) to the cyclodextrin is from 1:1 to 1:10.
